(19) United States
(12) Patent Application Publication
(10) Pub. No.: US 2011/0092970 A1
(43) Pub. Date: Apr. 21, 2011

(54) ELECTROSURGICAL DEVICE COMPRISING A SAFETY UNIT

(76) Inventors: Tobias Alberstetter, Mossingen (DE); Jürgen Hiller, Dettingen (DE); Achim Brodbeck, Metzingen-Neuhausen (DE)

(21) Appl. No.: 12/996,804

(22) PCT Filed: May 12, 2009
(86) PCT No.: PCT/EP09/03374

§ 371 Date: Dec. 8, 2010

(30) Foreign Application Priority Data
Jun. 11, 2008 (DE) 10-2008-027-788.6
Jul. 10, 2008 (DE) 10-2008-032.511.2

(51) Int. Cl. A61B 18/14

(52) U.S. Cl. 606/41

(57) ABSTRACT

An electrosurgical device for coagulating and/or cutting biological tissue that includes an instrument for application of high-frequency current, an activation switch, a high-frequency generator, and a safety unit. The instrument for application of the high-frequency current includes first and second branches having respective first and second jaw parts that are connected such that they are movable relative to one another. The safety unit interrupts the current supply to electrodes of the instrument when the jaw parts assume a position relative to one another that is not suitable for coagulation by means of the high-frequency current, e.g. when the jaw parts are open. Thus, erroneous operation of the device may be prevented.

### FIELD OF THE DISCLOSED EMBODIMENTS

The disclosed embodiments relate to an electrosurgical device, and in particular to and electrosurgical device including a safety unit.

### BACKGROUND

Electrosurgical instruments have been used for many years in high-frequency surgery to coagulate or cut biological tissue. In the case of coagulation, a high-frequency current is passed through the tissue to be treated, so that said tissue becomes changed due to protein coagulation and dehydration. The tissue contracts such that the vessels become closed and bleeding is staunched. Following coagulation, the tissue can be fully separated without the risk of severe bleeding, either with the aid of high-frequency current or by mechanical means.

Electrosurgical processes can be carried out by either monopolar or bipolar methods. With monopolar technology, the current path usually leads from a high frequency generator to an electrosurgical instrument, through the tissue to be treated, to a neutral electrode and from there back to the generator. However, bipolar instruments are known which, by contrast to monopolar instruments, have two electrodes for the application of the high-frequency current. With these instruments, the high-frequency current is conducted in via one electrode and conducted away via another. The current path between the two electrodes is therefore more readily calculable and does not follow long paths through the body of the patient.

DE 10 2006 042 985 A1 discloses a corresponding bipolar instrument having two electrodes, comprising two branches that are pivotably connected to one another. Disposed at the distal end of the branches are the electrodes, which are configured such that tissue can be grasped with them. Situated at the proximal end are handle devices for operating the branches. The electrosurgical instrument has a spacing element to create a defined minimum distance between the electrodes when the instrument is fully closed. There is a switch device for automatic activation of the high-frequency current. Use of the instrument is thus simplified, since the coagulation current is activated as soon as the branches are fully closed.

DE 102 05 093 A1 discloses a bipolar clamp which has catches at the proximal end of the branches. The catches serve to fix the bipolar clamp in a predetermined position. The catches also form a contact connection which activates the current. In the locked-in condition, coagulation takes place. The catch function of the bipolar clamp is practical in many applications, but has the disadvantage, on frequent opening and closing of the clamp, of being a hindrance. Furthermore, during locking-in, short undesirable current interruptions can occur.

The structure of these instruments is relatively complex and is associated with high production costs.

If, however, conventional electrosurgical instruments are considered, these often have a foot switch which ensures manual activation of the instrument. However, in this case, injury to the personnel or the patient can be caused by lack of caution or a faulty connection of the instrument. Activation of a corresponding electrosurgical instrument at the wrong time can result in burns or other injuries.

Thus, it is an object of the disclosed embodiments to provide an improved electrosurgical instrument.

### SUMMARY

Disclosed embodiments include an electrosurgical device including an instrument, such as a clamp or a pair of scissors, for cutting and/or coagulation of tissue with a high-frequency current, wherein the instrument has a first branch with a first jaw part and a second branch with a second jaw part and the branches are connected such that they are movable relative to one another, a high-frequency generator for generating a high-frequency current, an activation switch, connected to the high-frequency generator which, when actuated, feeds the high-frequency current from the high-frequency generator to the instrument, and a safety unit, which interrupts the current supply depending on the position of the jaw parts relative to one another.

Thus, in the disclosed embodiments, a conventional activation of the instrument by means of a hand switch or foot switch is possible. However, the safety unit prevents activation in cases where activation is undesirable. For this purpose, the safety unit determines the positions of the jaw parts relative to one another. In the simplest case, in an open condition of the jaw parts, the current flow through the safety unit is prevented, whereas in a closed position, current flow is allowed.

The instrument or clamp can include at least one terminal and at least one electrode for applying the high-frequency current, wherein the safety unit interrupts an electrical connection between the terminal and the electrode. Numerous mechanisms by which said current interruption can be carried out by the safety unit are conceivable. For example, suitable sensor devices can be mounted on the instrument to determine the position of the jaw parts and, depending thereon, possibly also taking account of other limit conditions, allow the high-frequency current to flow. It is, however, advantageous to design the safety unit to be as simple as possible. For this purpose, for example, a mechanical interruption of the current circuit can take place directly at the clamp.

The safety unit can include an angle detection device which determines an opening angle between the first and second jaw parts, wherein interruption of the current supply takes place at an opening angle of greater than 30°, or at an angle greater than 25°. The opening angle enables precise specification of the position of the jaw parts relative to one another. This position can be defined such that, in a closed or nearly closed position of the jaw parts, the angle is given as zero. Alternatively, given a flat configuration of the gripping surfaces of the jaw parts, the opening angle can be defined as zero if the surfaces lie parallel to one another. The angle calculation may be carried out by taking account of the rotational axis of the pivot joint connecting the branches.

The safety unit can include a first contact region and a second contact region to produce an electrical connection between at least one section on the first branch and at least one section on the second branch. The electrical contact regions form a switch which is mechanically integrated into the instrument. The switch is configured so as to assume the function of the safety unit.

The instrument or the clamp can include a pivot joint for connecting the branches, wherein the pivot joint comprises electrically insulating and electrically non-insulating portions to form the safety unit. The contact regions can therefore be part of the pivot joint or can be arranged in the immediate vicinity thereof. Since the pivot joint already forms a mechanical connection between the first and second branches, said pivot joint can advantageously be used for providing an electrical connection between the branches.

The instrument or clamp can be a bipolar clamp or pair of scissors comprising two electrodes and two high-frequency terminals. The safety unit can advantageously be used for bipolar instruments. Since the high-frequency terminals are usually arranged on one branch, the safety unit can be configured such that an advantageous routing of the electrical conductors is provided, wherein the electrodes arranged mutually opposed to each other are supplied with the high-frequency current.

At least one branch can include an insulation layer which insulates a first section of the branch against a second section of the branch, in order to form a first and second conductor path for the high-frequency current. In this way, the conductor tracks can advantageously be provided. The design effort involved is very small.

### BRIEF DESCRIPTION OF THE DRAWINGS

The disclosed embodiments will be described in greater detail, pointing out further features and advantages, by reference to the example embodiments illustrated in the drawings.

FIG. 1 shows a bipolar clamp with a pivot joint.

FIG. 2 shows a safety unit integrated into the pivot joint, in accordance with a disclosed embodiment.

FIG. 3 shows a cross-sectional view through the pivot joint along line III of FIG. 2.

FIG. 4 shows a cross-sectional view through the pivot joint along line IV of FIG. 2.

FIG. 5 shows a safety unit according to a disclosed embodiment formed by contact pins.

FIG. 6 shows a side view of the clamp of FIG. 5 from the direction of line VI.

FIG. 7 shows another embodiment, similar to the safety unit of FIG. 5.

FIG. 8 shows a side view of the clamp of FIG. 7 from the direction of line IX.

FIG. 9 shows a safety unit according to a disclosed embodiment with a pressure sensor.

FIG. 10 shows a safety unit according to a disclosed embodiment with a spring element.

FIG. 11 shows a safety unit according to a disclosed embodiment with a catch.

FIG. 12 shows a detailed cross-sectional view through the safety unit along line XII of FIG. 11.

FIG. 13 shows a safety unit according to a disclosed embodiment with a sliding contact,

FIG. 14 shows a safety unit according to a disclosed embodiment at the pivot joint of a clamp.

FIG. 15 shows a safety unit according to a disclosed embodiment at the pivot joint of a clamp.

FIG. 16 is a flow chart diagram of an electrosurgical device according to the disclosed embodiments.

### DETAILED DESCRIPTION

In the following description, the same reference signs are used for the same and similarly acting parts.

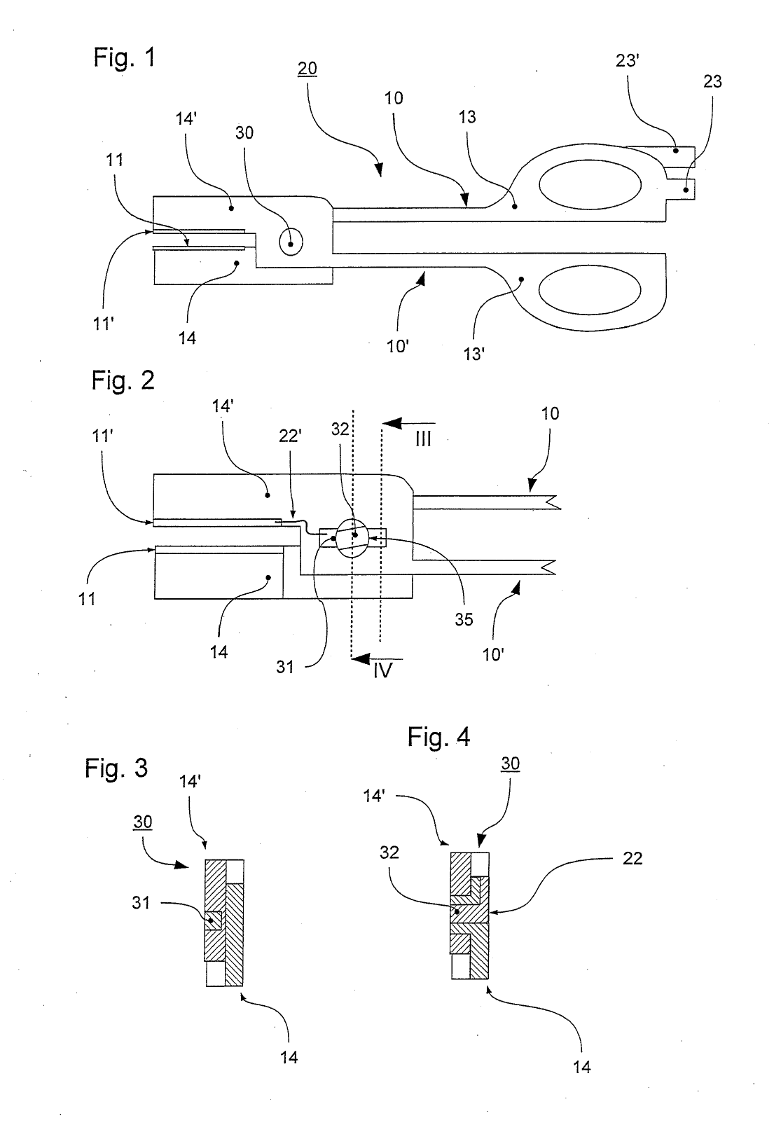

FIG. 16 shows the basic required components of the electrosurgical device according to the disclosed embodiments. These include a high-frequency generator 3 to provide a high-frequency current, an activating switch 5 (e.g. a foot switch or a hand switch) and a bipolar clamp 20. In order to carry out the coagulation procedure, the high-frequency current from the high-frequency generator 3 is applied to the bipolar clamp 20 on actuation of the activating switch 5. As shown in FIG. 1, for example, said clamp comprises a first high-frequency terminal 23 and a second high-frequency terminal 23′. The bipolar clamp 20 is made from a first branch 10 and a second branch 10′, connected to one another via a pivot joint 30. For better handling of the bipolar clamp 20, both high-frequency terminals 23, 23′ are situated at the proximal end of the first branch 10. The first branch 10 comprises a first handle part 13 and a first jaw part 14, wherein a first electrode 11 is arranged at the grip surface of the first jaw part 14. The second branch 10′ comprises a second grip part 13′ and a second jaw part 14′. Also arranged at the second jaw part 14′ is a second electrode 11′. For the coagulation of tissue, the current applied to the high-frequency terminals 23, 23′ is conducted via conductor paths to the corresponding electrode 11 or 11′.

In a first example embodiment (shown in FIGS. 2 to 4), the safety unit according to the invention is formed by contact regions 31, 32 in the pivot joint 30. The contact regions 31, 32 connect the current applied to the second high-frequency terminal 23′ through to the second branch 10′, particularly the electrode 11′ thereof.

In the plan view of the jaw parts 14, 14′ shown in FIG. 2, it is apparent that the second branch 10′ must be formed from electrically non-conductive material. Only a small region (second contact region 32) close to the joint axis 35 has electrically conductive material. The joint axis 35 also includes conductive and non-conductive material. The core of the joint axis 35 is made from conductive material. This conductive material extends, in sections, to the edge of the joint axis 35. The conductive region is designated the first contact region 31. The contact regions 31, 32 are arranged and configured such that they make contact with one another, depending on the position of the jaw parts 14, 14′ relative to one another. In the present example embodiment, the contact regions 31, 32, make contact when the jaw parts 14, 14′ are closed. This contact is maintained as far as a position in which the elongate jaw parts 14, 14′ enclose an angle between them which is approximately 30°. Thereafter, the non-conductive region of the joint axis 35 lies against the second jaw part 14′ in the first contact region 31. As can be seen from the cross-section through the first contact region 31 in FIG. 3, there is no direct contact between the first contact region 31 in the second jaw part 14′ and the first jaw part 14. Therefore, an electrical contact between the first jaw part 14 and the second jaw part 14′ can only be created via the pivot joint 30. Since the first branch 10 is made at least partially from electrically conductive material, it forms a first section 22 of a conductor path, which connects the second high-frequency terminal 22′ to the second contact region 32.

This section 22 can be an electrically conductive layer which extends along the longitudinal direction of the branch 10 and is separated from the remainder of the branch by an insulating layer.

FIG. 4 shows this section 22 of the conductor path together with the corresponding contacts. Once the high-frequency current has been passed through the pivot joint 30, a further section 22′ of a conductor path connects the first contact region 31 with the second electrode 11′.

The electrical connection between the high-frequency terminal 23′ and the electrode 11′ is made as follows: high-frequency terminal 23′, section 22 of the branch 10, second contact region 32, first contact region 31, section 22′, electrode 11′.

The electrical connection between the first high-frequency terminal 23 and the first electrode 11 can be carried out, for example, by means of a wire. This connection exists independently of the positions of the jaw parts 14, 14′ relative to one another.

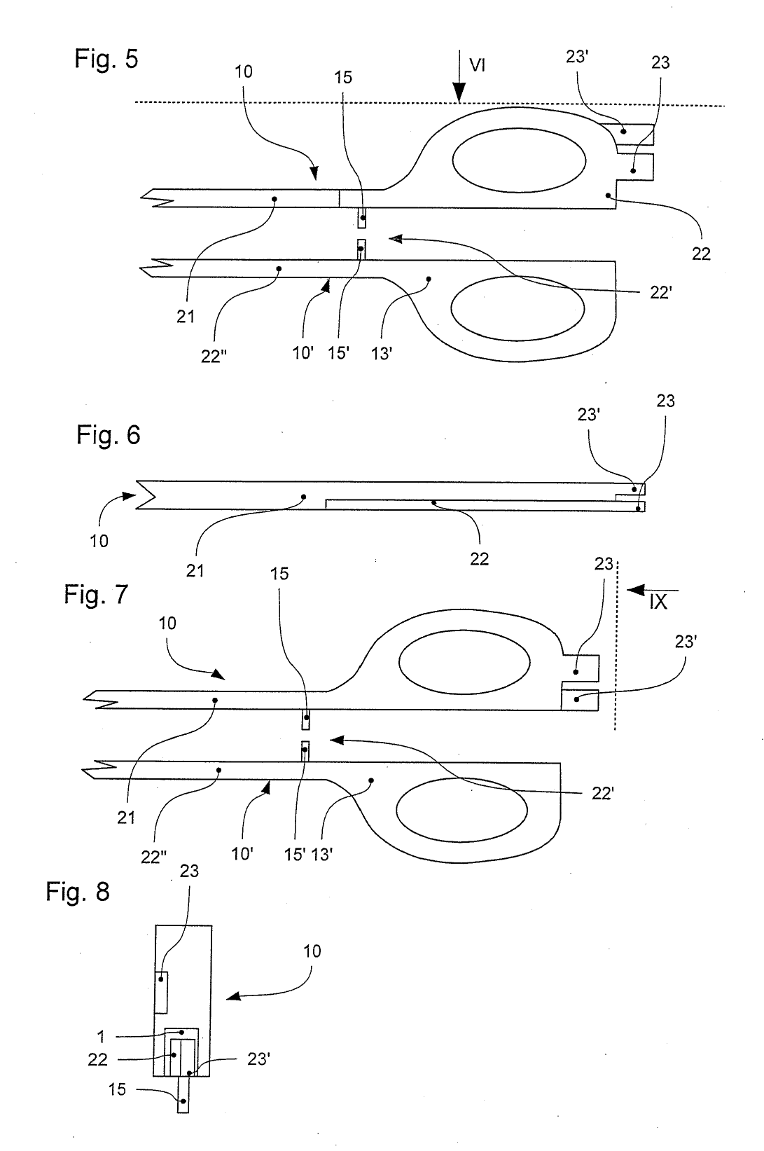

In a second example embodiment (see FIGS. 5 and 6), an electrical contact is made between the first branch 10 and the second branch 10′ via contact pins 15, 15′. These contact pins 15, 15′ make contact with one another depending on the position of the braches 10, 10′ to one another and form a section 22′ of a conductor path between the high-frequency terminal 23′ and the second electrode 11′. A further section 22 of this conductor path is formed by a layer which is electrically separate from the remainder of the first branch 10, and is arranged on the first branch 10.

The branches 10, 10′ according to the second example embodiment are made as far as possible from electrically conductive material. As FIG. 6 shows, part of the first branch 10 forms a section 21 of the first conductor path for the first electrode 11. The second conductor path for the second electrode 11′ comprises the section 22, the contact pins 15, 15′ (that form section 22′) and the entire second branch 10′, particularly section 22″, which is in direct electrical contact with the second electrode 11′. In this second example embodiment, the pivot joint 30 comprises a non-conductive, purely mechanical connection between the first branch 10 and the second branch 10′.

It is obvious that, depending on the design of the contact pins 15, 15′ the activation of the high-frequency current is adjustable depending on the position of the jaw parts 14, 14′ relative to one another. It can be advantageous to insulate the contact pins 15, 15′ that are welded to the corresponding branches 10 and 10′ such that only a narrow contact region remains at the tips of the contact pins 15, 15′.

In a third example embodiment, the first branch 10 has a recess in the lower section thereof along the longitudinal axis thereof. Arranged in this recess is an insulation layer 1, which forms an electrical barrier layer between a section 22 of the second conductor path arranged there and the remainder of the branch 10. The contact pin 15 directly adjoins said section 22 of the conductor path. FIG. 8 shows a plan view of the proximal end of the branch 10. The section 22 in the recess is welded to the contact pin 15. An electrical connection is thus formed. The second terminal 23′ is also in direct electrical connection with this section 22. The first electrical terminal 23 is attached to the rear side of the branch 10 and, together with the remainder of the first branch 10, forms the first conductor path for the first electrode 11.

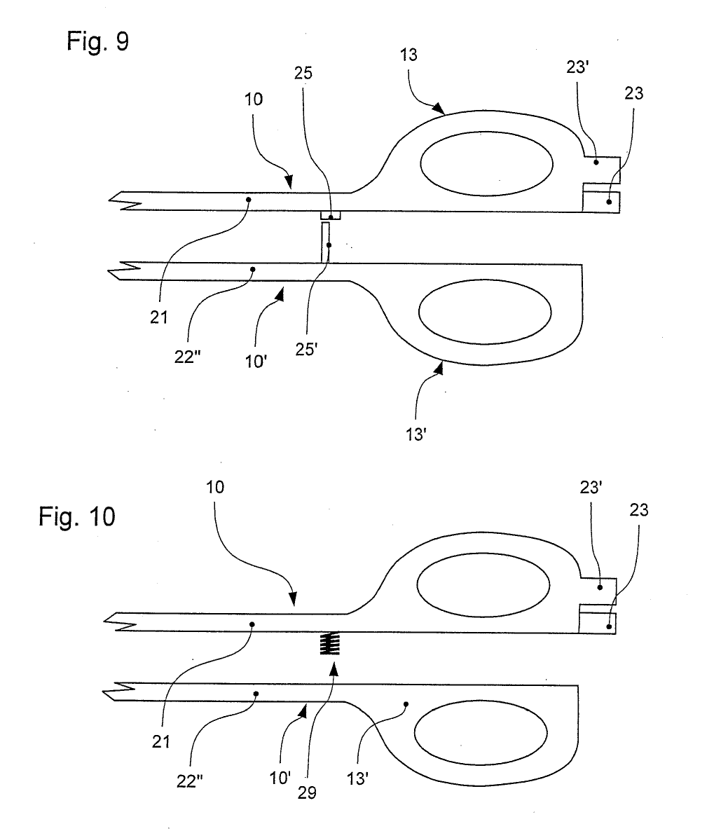

In a fourth example embodiment, the safety unit is a pressure sensor 25 (FIG. 9). This pressure sensor 25 is arranged at the underside of the first branch 10. When the handle parts 13, 13′ are pressed together, a suitably arranged pin 25′ 5 on the second branch 10′ presses against the pressure sensor 2. Depending on whether the pressure sensor 25 registers a pressure or not, the electrical connection is made between the second high-frequency terminal 23′ and the second electrode 11′. In this example embodiment, the electrical connection between a high-frequency terminal 23 and the first electrode 11 is formed by suitably arranged conductors. This connection exists irrespective of the position of the branches 10, 10′ relative to one another.

In a fifth example embodiment, the contacts 15, 15′, as per the second and third example embodiments, are replaced by a spring element 29. The spring element 29 can create a flexible contact between the branches 10, 10′ (see FIG. 10).

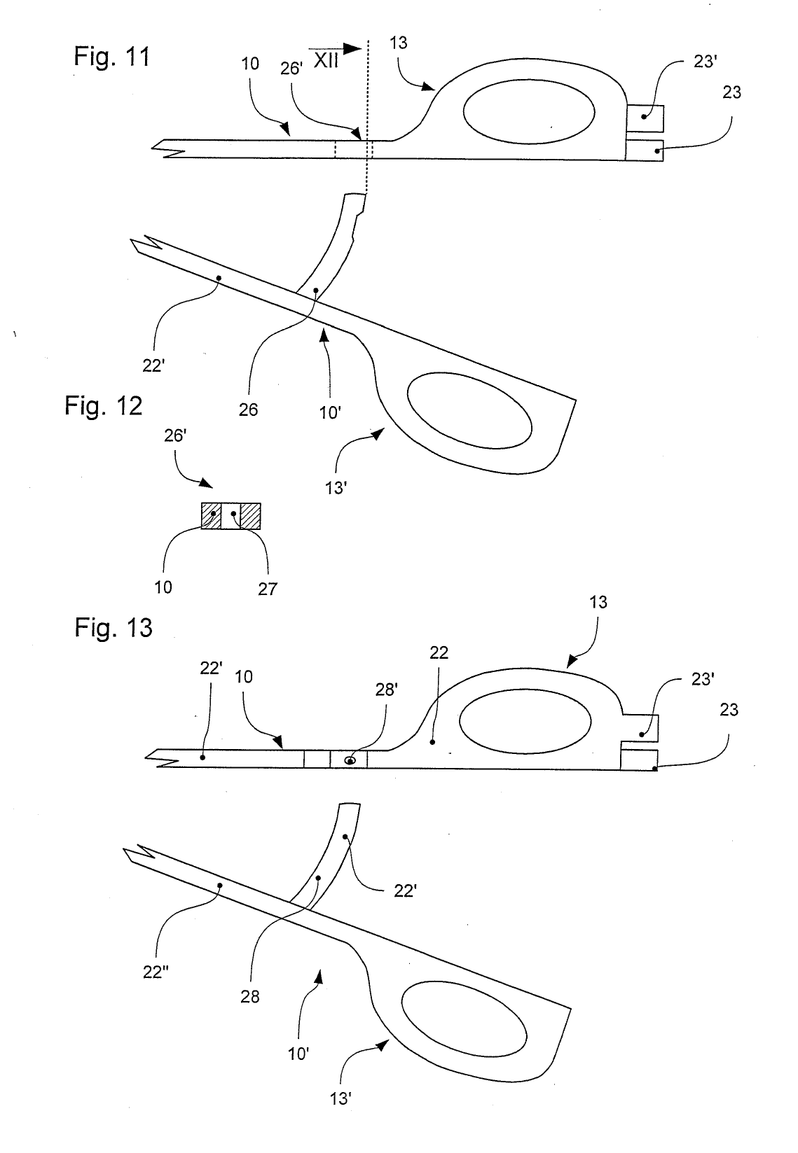

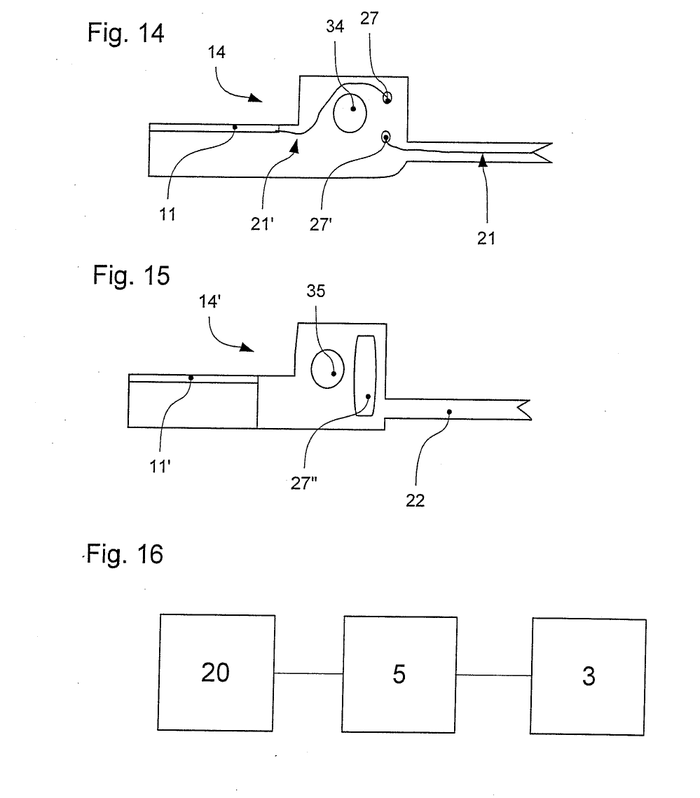

In a sixth example embodiment (see FIG. 11), the bipolar clamp 20 comprises a catch 26 and a corresponding catch opening 26′. The catch 26 is arranged on the second handle part 13′ and extends in a direction toward the first handle part 13. The catch 26 is curved toward the distal direction and has a narrowing in the upper section thereof. After a deformation of the catch 26, said narrowing can engage in the catch opening 26′, which extends transversely to the longitudinal axis of the first handle part 13 and through said handle part. As FIG. 12 shows, the catch opening 26′ has a contact region 27 at the proximal side thereof. This contact region 27 is in electrical contact with the second high-frequency terminal 23′. The remainder of the catch opening 26′ is covered with electrically insulating material. The contact region 27 is therefore insulated against the remainder of the first branch 10. When the catch 26 engages in the catch opening 26′, an electrical connection comes about between the second branch 10′ and the second high-frequency terminal 22′. A conductor path comes into existence, leading from the second high-frequency terminal 23′ to the contact region 27 and via the catch 26 and the second branch 10′ to the second electrode 11′.

In a seventh example embodiment (see FIG. 13), the electrical contact is made between the first and second branches 10, 10′ via a sliding contact. The branch 10 of the seventh example embodiment has a multi-layered structure, as previously described by reference to FIG. 6. The coatings or layers comprise a first section 21 of a conductor path for the first high-frequency terminal 23 and a further section 22 for the second high-frequency terminal 23′. The individual sections 21, 22 are insulated relative to one another. A contact ball 28′ is mounted in the further section 22. The position of the contact ball 28′ is chosen such that, from a particular opening angle of the branches 10, 10′, said contact ball contacts a projection 28 arranged on the second branch 10′. The sections 22, 22′ (projection 28) and 22″ (branch 10′) therefore constitute an interruptible conductor path for the second electrode 11′.

It should be obvious for a person skilled in the art that various possibilities exist for producing a similar sliding contact between the branches 10, 10′. It should also be obvious for a person skilled in the art how the projection 28 should be configured in order to vary the activation of the safety unit, depending on the angle of the jaw parts 14, 14′.

In a last example embodiment, contact regions 27, 27′ are arranged close to the pivot joint 30 at the inner side (the side facing toward the second jaw part 14′) of the first jaw part 14. The first contact region 27 is connected to the electrode 11 via a section 21′ of a conductor path, whilst the second contact region 27′ is connected via a further section 21 of a conductor path to the first high-frequency terminal 23. The other sections of the first branch 10 are formed from electrically non-conductive material or have at least one insulation layer, so that no disturbance of the functionality of the bipolar clamp 20 comes about.

FIG. 15 shows the inner side of the second jaw part 14′. A large-area third contact region 27″ is arranged close to the joint axis 35 of the pivot joint 30. Said contact region 27″ is insulated against other sections of the second jaw part 14′. As soon as the jaw parts 14, 14′ assume a position relative to one another which corresponds to an angle smaller than 25°, the first contact regions 27, 27′ contact the third contact region 27″. The contact region 27″ creates an electrical connection between the first contact region 27 and the second contact region 27′. The high-frequency current can flow unhindered to the first electrode 11. The second electrode is supplied via a conductive section 22 in the second branch 10′.

It should be noted at this point that all the aforementioned parts are claimed as essential to the invention both alone and in any combination, particularly the details shown in the drawings. Amendments thereof are the common practice of persons skilled in the art.

1-7. (canceled)

8. An electrosurgical device, comprising:
an instrument for cutting and/or coagulation of tissue with a high-frequency current, wherein the instrument has a first branch with a first jaw part and a second branch with a second jaw part and the first and second branches are connected such that they are movable relative to one another;
a high-frequency generator for generating a high-frequency current;
an activation switch connected to the high-frequency generator that, when actuated, causes the high-frequency current to flow from the high-frequency generator to the instrument; and
a safety unit, configured to selectively interrupt the high-frequency current depending on the position of the first and second jaw parts relative to one another.

9. The electrosurgical device according to claim 8, wherein the instrument comprises at least one terminal and at least one electrode for applying the high-frequency current, and wherein the safety unit interrupts the high-frequency current by interrupting an electrical connection between the terminal and the electrode.

10. The electrosurgical device according to claim 8, wherein the safety unit comprises an angle detection device for determining an opening angle between the first and second jaw parts, and wherein the high-frequency current is interrupted when the opening angle is greater than about 30°.

11. The electrosurgical device according to claim 10, wherein the high-frequency current is interrupted when the opening angle is greater than about 25°.

12. The electrosurgical device according to claim 8, wherein the safety unit comprises a first contact region and a second contact region to produce an electrical connection between at least one section on the first branch and at least one section on the second branch.

13. The electrosurgical device according to claim 8, wherein the instrument comprises a pivot joint for connecting the first and second branches, wherein the pivot joint comprises electrically insulating and electrically non-insulating portions to form the safety unit.

14. The electrosurgical device according to claim 8, wherein the safety unit comprises a first contact pin and a second contact pin disposed on respective ones of said first and second branches.

15. The electrosurgical device according to claim 8, wherein the safety unit comprises a pressure sensor provided on one of the first and second branches and a corresponding pin on the other of the first and second branches.

16. The electrosurgical device according to claim 8, wherein the safety unit comprises a spring provided on one of the first and second branches.

17. The electrosurgical device according to claim 8, wherein the safety unit comprises a catch provided on a handle of one of the first and second branches and a corresponding catch opening in a handle of the other of the first and second branches.

18. The electrosurgical device according to claim 8, wherein the safety unit comprises a sliding contact provided on a handle of one of the first and second branches and a corresponding contact ball within an opening in a handle of the other of the first and second branches.

19. The electrosurgical device of claim 8 wherein the instrument is a clamp.

20. The electrosurgical device according to claim 19, wherein the instrument is a bipolar clamp comprising at least two electrodes and two high-frequency terminals.

21. The electrosurgical device of claim 8 wherein the instrument is a pair of scissors.

22. The electrosurgical device according to claim 21, wherein the pair of scissors comprises at least two electrodes and two high-frequency terminals.

23. The electrosurgical device according to claim 8, wherein at least one of the first and second branches comprises an insulation layer which electrically insulates a first section of the at least one branch against a second section of the at least one branch, in order to form a first and second conductor path for the high-frequency current.
